(19) United States
(12) Patent Application Publication
(10) Pub. No.: US 2025/0308654 A1
(43) Pub. Date: Oct. 2, 2025

(54) STANDARDIZED PSYCHIATRIC INTERVIEW SYSTEM

(71) Applicant: MindfulActs LLC, Kathleen, GA (US)

(72) Inventor: Sheldon Brown, Kathleen, GA (US)

(21) Appl. No.: 19/091,477

(22) Filed: Mar. 26, 2025

Related U.S. Application Data
(60) Provisional application No. 63/570,999, filed on Mar. 28, 2024.

(51) Int. Cl. G16H 10/60; G16H 10/20

(52) U.S. Cl. CPC G16H 10/60; G16H 10/20

(57) ABSTRACT

Methods, systems, and computer-readable storage media for providing a streamlined and standardized psychiatric interview process Psychiatric interview information from a client is received over a network and stored in a database. It is determined whether the received psychiatric interview information for the client is complete, and upon determining that the psychiatric interview information for the client is complete, the received psychiatric interview information for the client is formatted into a standardized patient notes document. The standardized patient notes document for the client is then provided to a provider associated with the client over the network.

### CROSS-REFERENCE TO RELATED APPLICATIONS

This application claims the benefit of U.S. Provisional Application No. 63/570,999 filed on Mar. 28, 2024, and entitled “STANDARDIZED PSYCHIATRIC INTERVIEW SYSTEM,” the entire disclosure of which is hereby incorporated herein by this reference.

### BACKGROUND

The psychiatric interview is a crucial step in the diagnosis and treatment of mental health conditions. However, traditional psychiatric interview processes can be time-consuming and often lead to delays in receiving appropriate care.

There is a need for a user-friendly platform that addresses these challenges by providing a streamlined interview process and an enhanced overall client experience.

### BRIEF SUMMARY

The present disclosure relates to technologies for providing a standardized psychiatric interview system and application that streamlines the psychiatric interview process enabling clients to initiate their treatment sooner as well as facilitating a community-based approach to psychiatry. According to some embodiments, one method for method of conducting a standardized psychiatric interview with a client online includes receiving psychiatric interview information from a client over a network and storing the received psychiatric interview information in a database. It is determined whether the received psychiatric interview information for the client is complete, and if the received psychiatric interview information for the client is not complete, the receiving, storing, and determining steps are repeated until the psychiatric interview information for the client is complete. Upon determining that the psychiatric interview information for the client is complete, the received psychiatric interview information for the client if formatted into a standardized patient notes document, which is then provided to a provider associated with the client over the network.

According to further embodiments, a computer-readable medium is encoded with computer-executable instructions that, when executed by a processor of a standardized psychiatric interview system, cause the standardized psychiatric interview system to receive psychiatric interview information from a client over a network and store the received psychiatric interview information in a database. It is determined whether the received psychiatric interview information for the client is complete, and upon determining that the received psychiatric interview information for the client is complete, the received psychiatric interview information for the client is formatted into a standardized patient notes document. The standardized patient notes document for the client is then provided to a provider associated with the client over the network.

According to further embodiments, a standardized psychiatric interview system comprises a database and a processor. The processor is operably connected to the database and configured to, upon receiving psychiatric interview information from a client over a network, store the received psychiatric interview information in the database. It is determined whether the received psychiatric interview information for the client is complete, and upon determining that the psychiatric interview information for the client is complete, the received psychiatric interview information for the client is formatted into a standardized patient notes document. The standardized patient notes document for the client is then provided to a provider associated with the client over the network.

These and other features and aspects of the various embodiments will become apparent upon reading the following Detailed Description and reviewing the accompanying drawings.

### BRIEF DESCRIPTION OF THE DRAWINGS

In the following Detailed Description, references are made to the accompanying drawings that form a part hereof, and that show, by way of illustration, specific embodiments or examples. The drawings herein are not drawn to scale, and any measurements provided are shown to provide a relative size context and are not intended to be limiting. Like numerals represent like elements throughout the several figures.

FIG. 1 is a system diagram showing components of an illustrative standardized psychiatric interview system, according to embodiments presented herein.

FIG. 2 is a block diagram showing software modules and components of the standardized psychiatric interview system, according to further embodiments.

FIG. 3 is a use case diagram for the standardized psychiatric interview system, according to further embodiments.

FIG. 4 is a flow chart showing an exemplary routine facilitating client and provider interaction with standardized psychiatric interview system, according to embodiments presented herein.

FIGS. 5A-5Y are GUI diagrams showing an exemplary user interface used by clients to interact with the standardized psychiatric interview system, according to embodiments presented herein.

FIG. 6 is a block diagram showing an exemplary computing and software architecture for computing devices described herein.

FIG. 7 is an inventory list of database tables and stored procedures for storing data supporting the standardized psychiatric interview system, according to embodiments presented herein.

### DETAILED DESCRIPTION

The present disclosure relates to technologies for providing a streamlined and standardized psychiatric interview process. According to embodiments described herein, computer systems and software applications (referred to herein variously as “the MindFit Psychiatric Application” or “the application”) may be implemented that provide both patients (also referred to herein as “clients”) and doctors (also referred to herein as “providers”) a convenient and efficient solution to the psychiatric interview process. Clients may utilize the MindFit Psychiatric Application to complete various aspects of the psychiatric interview at their convenience, either at home or prior to seeing a psychiatric provider. The collected information is then sent to the provider in a psychiatric note format, enabling the client to be seen promptly after completing the questionnaire. The application may further incorporate videos to bridge the gap between encounters and enhance the user experience. Additionally, the application may integrate AI technology to provide clients with community resources, such as therapists, support groups, reading materials, and workbooks, before their psychiatric appointment.

The MindFit Psychiatric Application is designed to facilitate a community-based approach to psychiatry and enable clients to initiate their treatment sooner. According to various embodiments, the application may offer a number of features and functions that provide advantages over traditional psychiatric interview methods:
Convenient Completion: Clients can complete various aspects of the psychiatric interview at their convenience, eliminating the need for in-person visits solely for this purpose. This flexibility allows clients to engage with the application from the comfort of their own homes, saving time and reducing barriers to care.
Prompt Access to Care: Once a client has submitted the completed questionnaire, the psychiatric provider receives the information in a psychiatric note format. This enables the provider to review the client's responses before the appointment, allowing for a more focused and productive session. Consequently, clients can be seen promptly after completing the questionnaire, minimizing waiting times and expediting the treatment process.
Video Integration: The application incorporates videos throughout the questionnaire to provide greetings and explanations for key aspects. This feature bridges the gap between encounters and enhances the user experience, making the process more engaging and informative.
AI Integration: The application utilizes AI technology to help organize the information gathered from the questionnaire to generate legible notes for the provider. This streamlines the process and ensures that all relevant details are captured accurately. In addition, AI can be utilized to incorporating the bio-psychosocial information of a client to help them find therapeutic resources in their specific area. By leveraging AI, we can identify and recommend therapies and other resources that are readily available throughout the community but may be unknown to clients. This includes access to therapists, support groups, reading materials, and workbooks that can aid in their treatment journey. By providing these resources before the psychiatric appointment, clients can begin their treatment sooner and feel supported throughout the process as well as bridge the gap between clients and valuable resources that can address their emotional concerns effectively.
Enhanced Efficiency: By allowing clients to complete the questionnaire remotely, the application saves time for both clients and providers. This efficiency enables providers to see more clients and dedicate more time to meaningful interactions during appointments.
Improved Client Experience: The user-friendly interface, video integration, and access to community resources make the application engaging and supportive. Clients feel empowered and informed, leading to increased satisfaction and better treatment outcomes.
Community-Based Approach: The application fosters a community-based approach to psychiatry by connecting clients with valuable resources before their appointment. This approach encourages collaboration, self-help, and early intervention, ultimately improving overall mental health outcomes.
Unique User Interface: A user interface that is innovative, intuitive, and tailored specifically for the psychiatric interview process.
Seamless Integration: The application offers seamless integration with existing electronic medical records (“EMR”) systems. This ensures efficient data transfer and compatibility with existing workflows, making it fairly simple for healthcare providers to adopt the application.
AI-Powered Decision Support: The application incorporates artificial intelligence algorithms that can analyze user responses and provide intelligent recommendations or suggestions. This will involve using machine learning techniques to identify patterns, predict outcomes, or offer personalized support options.
Video Integration: Including videos within the application that provide guidance, explanations, or educational content can enhance the user's experience and improve engagement. This feature could involve creating original video content or partnering with experts in the field to provide high-quality visual resources.
Community Resources: The application is integrated with community resources, such as therapist directories, support groups, or educational materials. This helps users access additional support and information beyond the psychiatric interview, promoting a holistic approach to mental health care.
Data Security and Privacy: The application implements robust security measures to protect user data and ensure compliance with privacy regulations. This includes encryption, secure data storage, and adherence to HIPAA.
Customization and Scalability: The application and system design allow customization options for healthcare providers to tailor the application to their specific needs and is scalable, accommodating a large number of users and expanding functionality as needed.

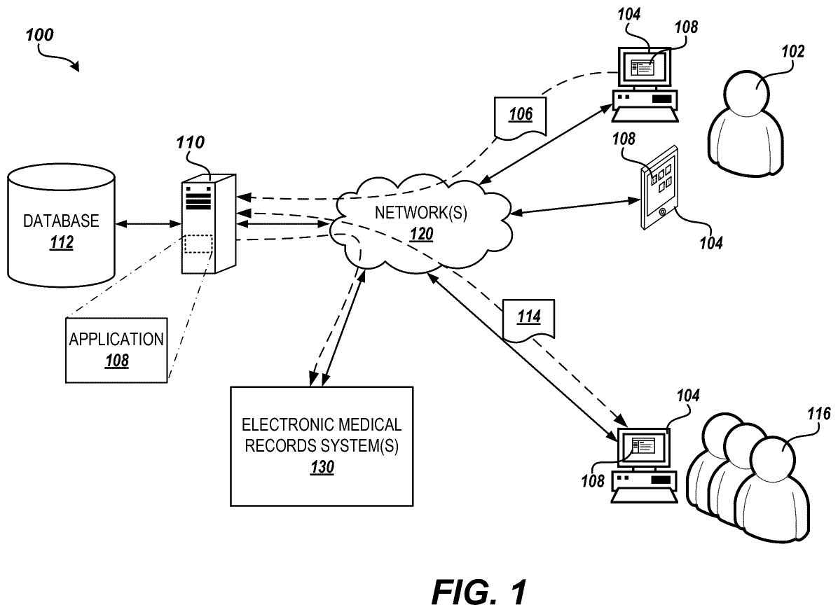

FIG. 1 shows an exemplary standardized psychiatric interview system 100 within which the MindFit Psychiatric Application may be implemented, according to some embodiments. The standardized psychiatric interview system 100 allows patients or “clients” 102 to provide psychiatric interview information 106 for providers. For example, a client 102 may utilize a computing device 104 for patient registration at home before an appointment with a provider or while sitting in the waiting room utilizing a provider-provided tablet to provide the psychiatric interview information 106, including answers to psychiatric interview questions, pathology, basic patient information, family history, medical history, and the like.

The computing device 104 may represent any computing device, components, or systems that allow the client 102 to access the functions of the standardized psychiatric interview system 100, such as a smartphone, tablet, laptop, or desktop computer executing an Internet browser application. The computing device 104 is connected to one or more cloud-based servers 110 over one or more networks 120. The network(s) 120 may comprise any combination of networking infrastructure that connects the computing device 104 to the cloud-based server(s) 110, such as 5G or LTE cellular data networks, LANs, WANs, MANs, and/or the Internet. The cloud-based server(s) 110 may represent virtualized computing resources available in the cloud, such as those provided by Amazon Web Services (“AWS”), Google Cloud Platform (“GCP”), or Microsoft Azure. In further embodiments, the cloud-based server(s) 110 may represent one or more of conventional web servers, application servers, and/or other application hosting environments executing on one more server computers.

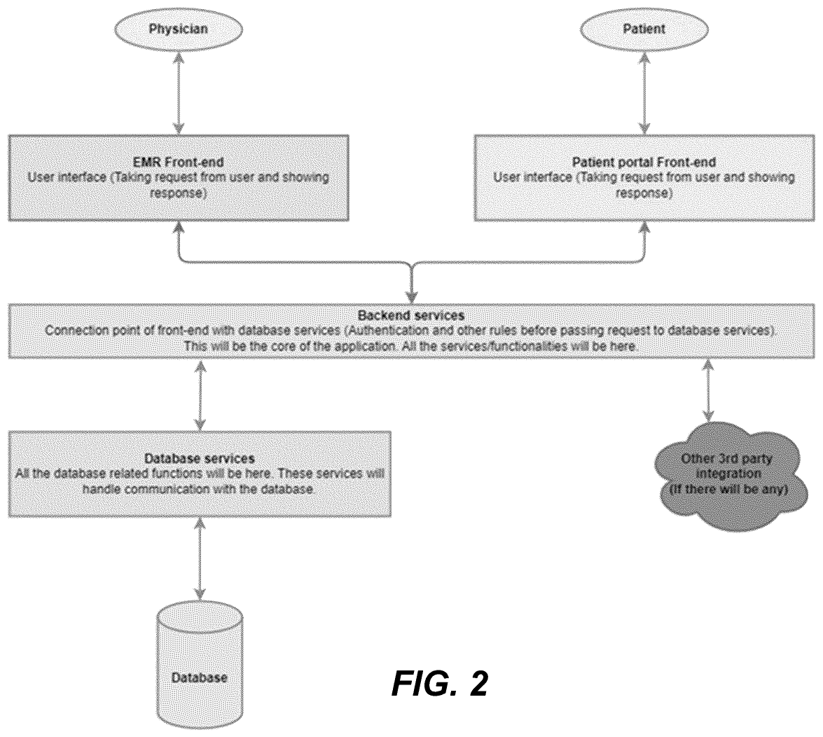

According to embodiments, the computing device 104 and cloud-based server(s) 110 cooperatively execute software components and modules collectively forming the MindFit Psychiatric Application 108. In various embodiments, the MindFit Psychiatric Application 108 may comprise of one or more software modules and components, including an electronic medical records (“EMR”) frontend, a patient-portal frontend, backend services, and database services, as illustrated in FIG. 2, each responsible for implementing various aspects of functionality described herein.

In further embodiments, the MindFit Psychiatric Application 108 stores the provided psychiatric interview information 106 from the client 102 in a database 112. The database 112 may represent any data storage facility known in the art. According to some embodiments, the client 102 may provide their psychiatric interview information 106 over multiple “sessions” of usage of the application 108, as well as edit previously provided information. The MindFit Psychiatric Application 108 may encrypt the psychiatric interview information 106 stored in the database 112 and restrict access to the information the client 102, their provider(s), and approved administrative personnel, in accordance with HIPPA and other medical data security standards and practices.

According to embodiments, the MindFit Psychiatric Application 108 takes the provided psychiatric interview information 106 and make it available to providers for purposes of treating clients 102. For example, the psychiatric interview information 106 may be retrieved by MindFit Psychiatric Application 108 from the database 112, formatted into a document comprising a “patient notes” format, and make the patient notes document 114 accessible to the provider, admins, medical residents, or other authorized personnel (collectively shown at 116) on corresponding computing devices 104. The psychiatric interview information 106, patient notes document 114, and other information relevant to clients 102 may further be made available to third-party EMR systems 130 over the network(s) 120.

According to further embodiments, the MindFit Psychiatric Application 108 may also incorporate the following features:
Patients can fill in their encounter questionnaire. After filling, the questionnaire will go to Provider and patient will not have access to that.
Patients can enter their current medication and medical history.
Patients can schedule appointment with provider.
Medical residents can view patient details.
Provider can also view patient details. Limited to their facility only.
Provider can set up their schedule (set their availability for patients).
Application supports multilingual features.
Application provides facilities to translate user input data.
Application incorporates telehealth features.

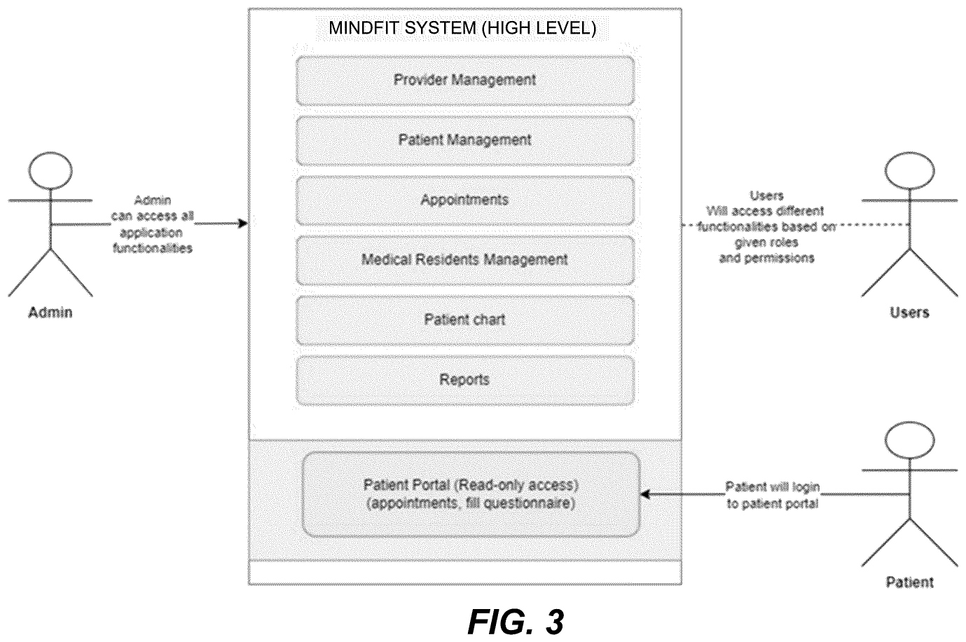

In further embodiments, the MindFit Psychiatric Application 108 may support the functionality for the following roles, as shown in FIG. 3:
Admin—Full access to everything, including managing provider's records (Add/Update/Deactivate).
Providers can login, manage medical residents (Add/Update/Deactivate), view patient demographics, view questionnaire filled by patient, change password, and update profile. Providers can access patient's (client 102) data for patients of only their facility. (Facility will be created by Admin). Providers can also manage medical resident's records.
Medical residents can login, view patient demographics, view questionnaires filled by patient, change password, and update profile. Can access patient's data of patients of only their facility. (Facility will be created by Admin)
Patients/Clients can register, log in, fill questionnaire(s), schedule appointments, change password, and update profile. (Choose facility to send questionnaire.)

Information maintained by the MindFit Psychiatric Application 108 in the database 112 for various users include:
Medical resident's information—First name, Last name, email (unique to the application), phone (unique to application), address line 1, address line 2, city, state, zip. Profile photo. Photo id, NPI.
Provider information—First name, Last name, email (unique to the application), phone (unique to application), NPI, address line 1, address line 2, city, state, zip. Photo id, NPI.
Patient information—First name, Last name, email (unique to the application), phone (unique to application), address line 1, address line 2, city, state, zip, DOB, Insurance information? (Yes—primary and secondary), profile photo, photo id., Family and social history of patient, Allergies.

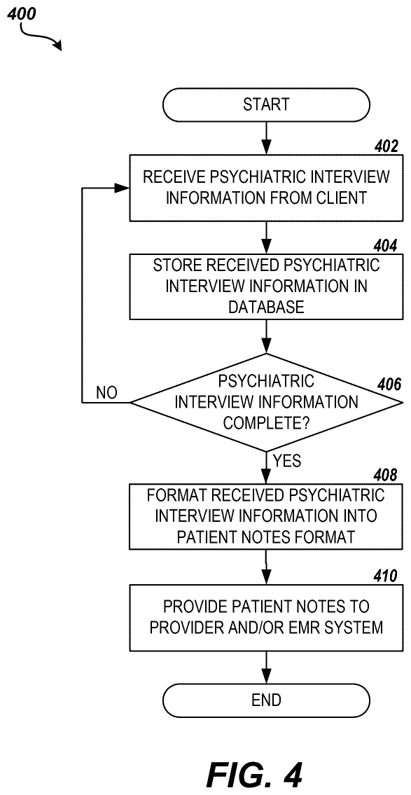

FIG. 4 illustrates one routine 400 for providing a streamlined and standardized psychiatric interview process, according to embodiments described herein. In some embodiments, the steps of the routine 400 may be performed by some combination of computing devices 104, cloud-based server(s) 110, and/or other computing devices, components, and modules of the standardized psychiatric interview system 100. The steps of the routine 400 may be directed by the MindFit Psychiatric Application 108 residing/executing on the computing devices 104 and cloud-based servers 110, for example.

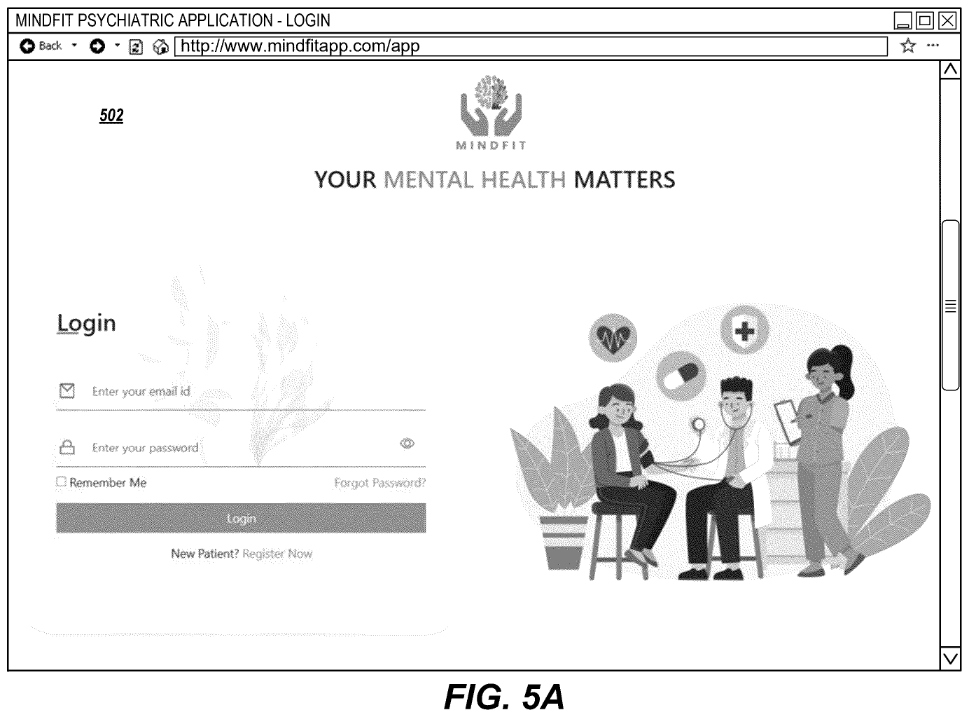

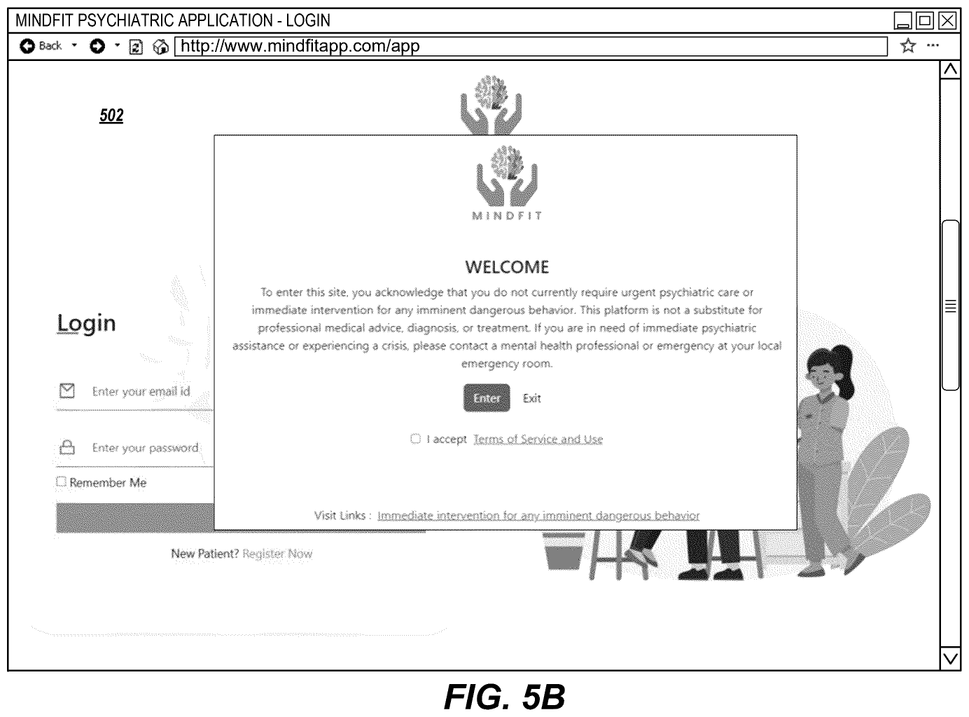

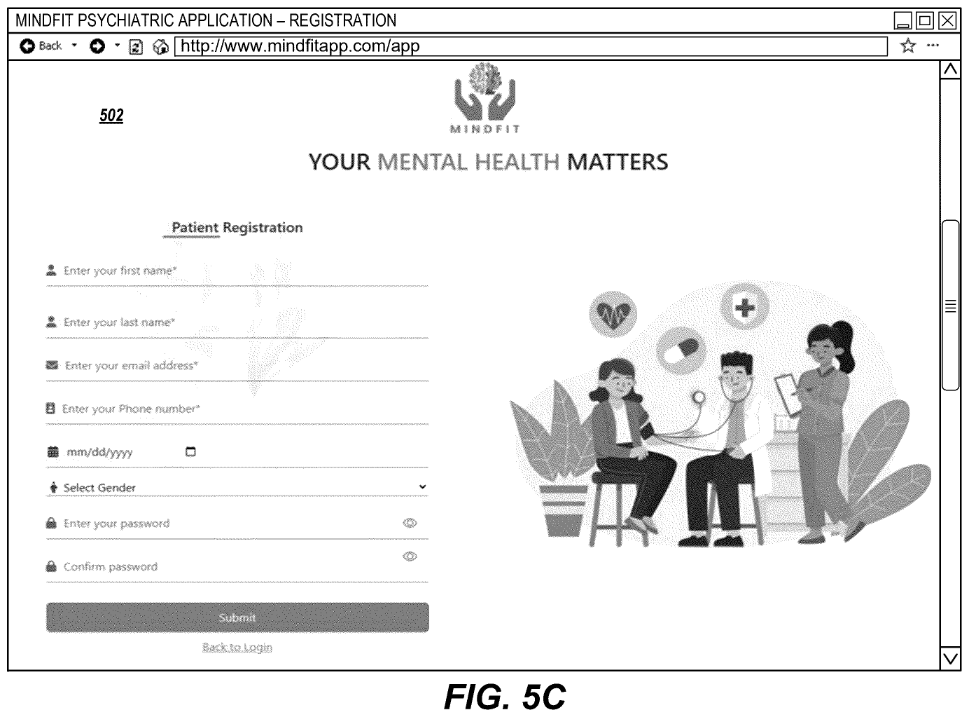

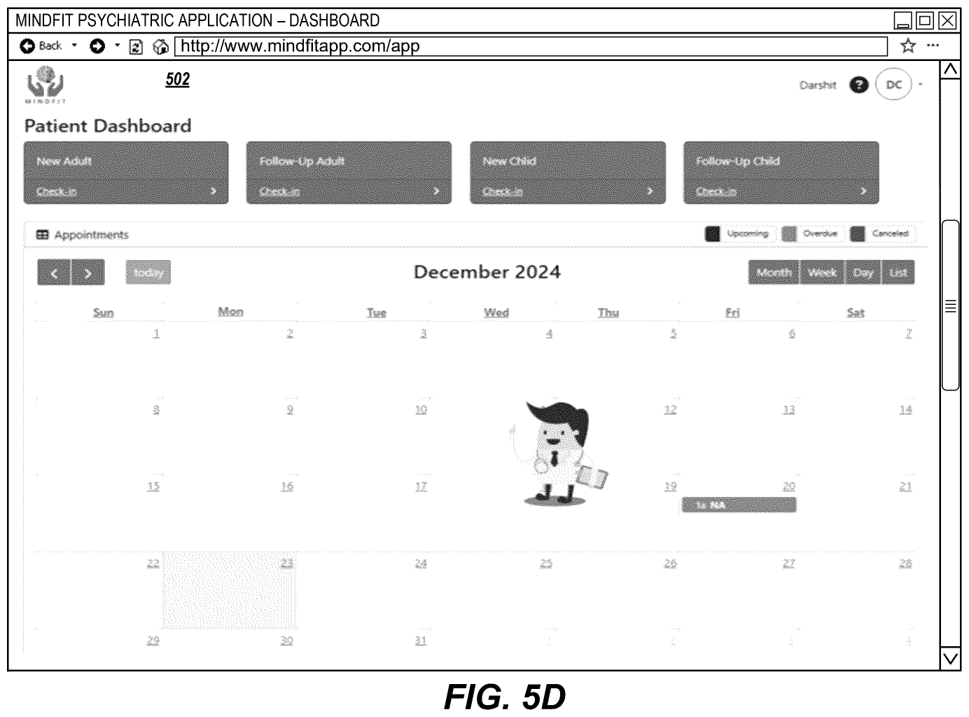

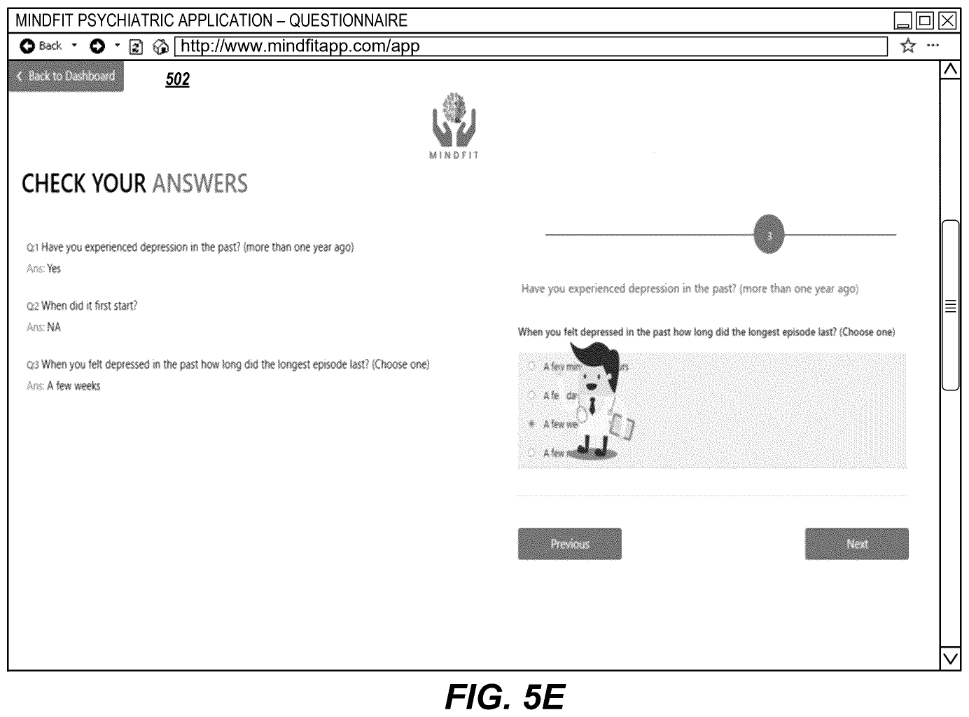

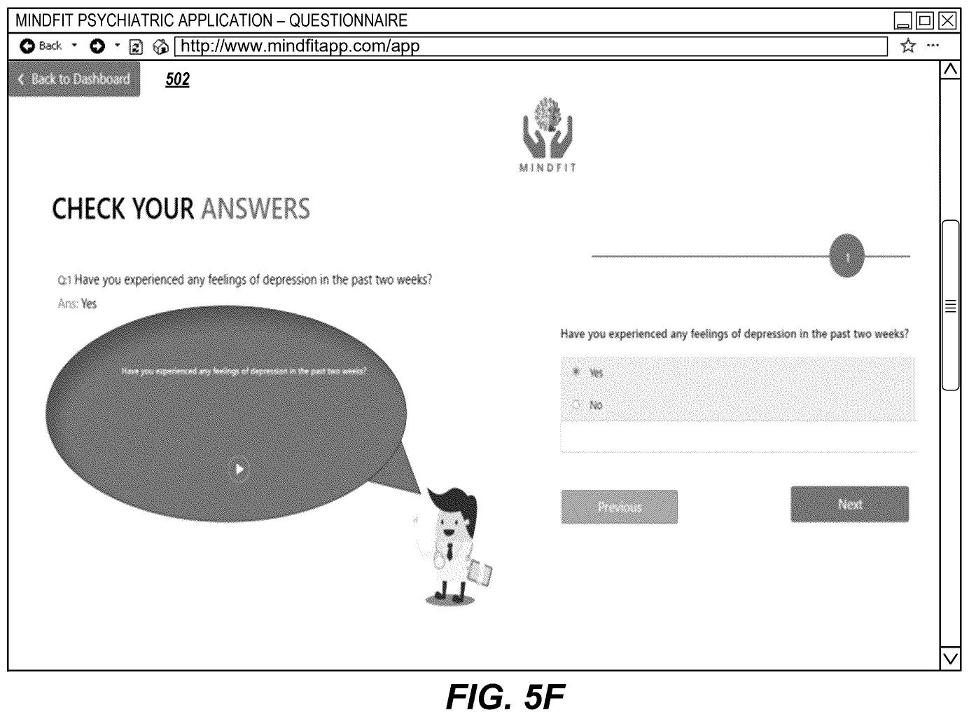

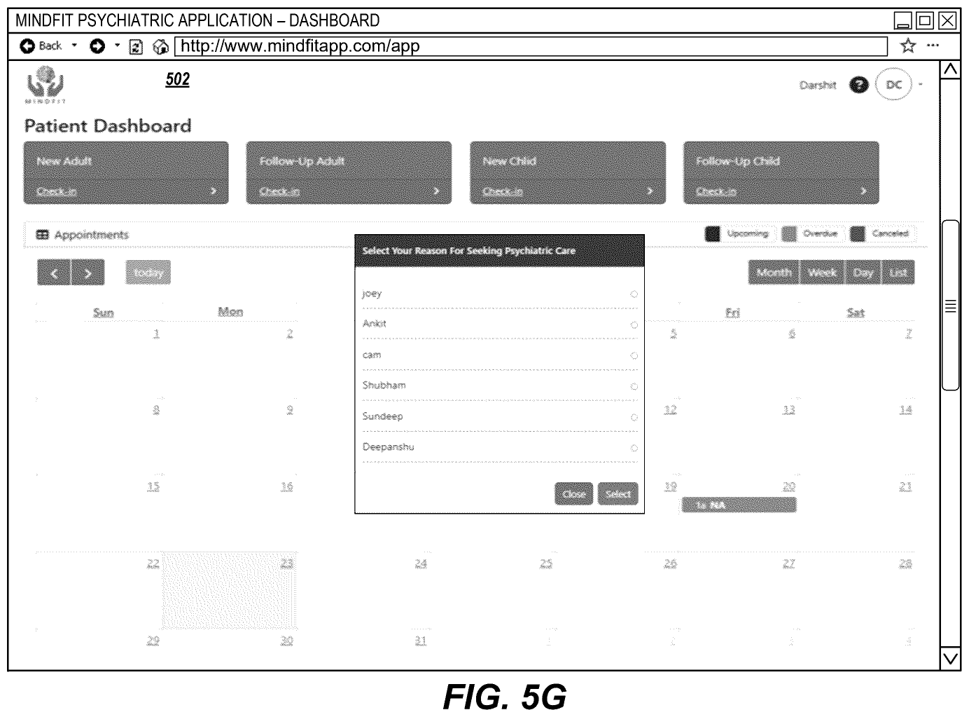

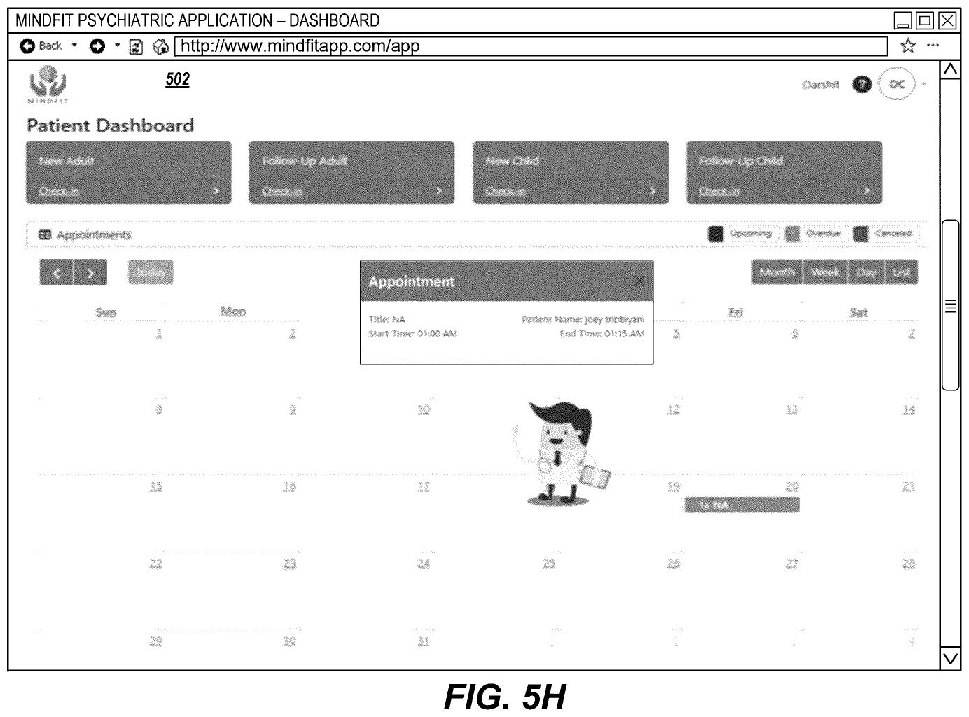

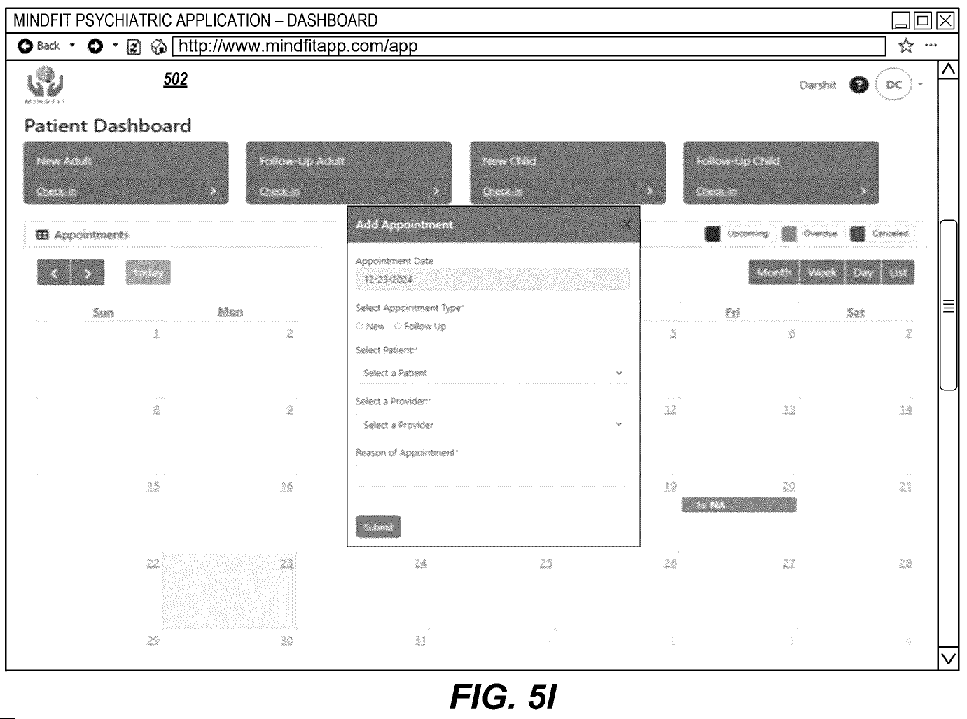

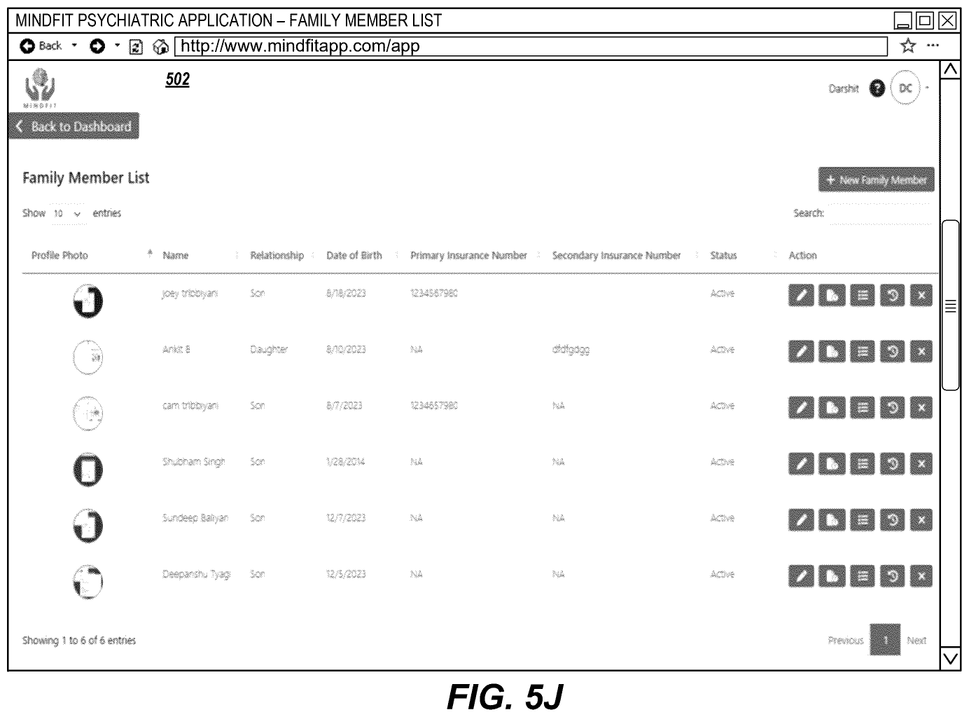

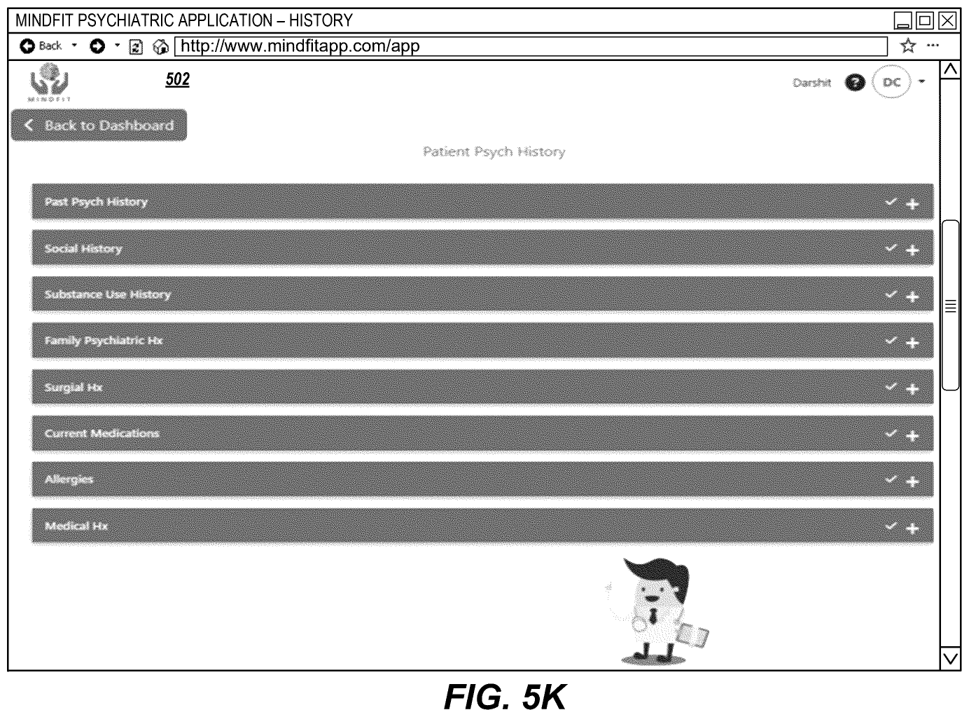

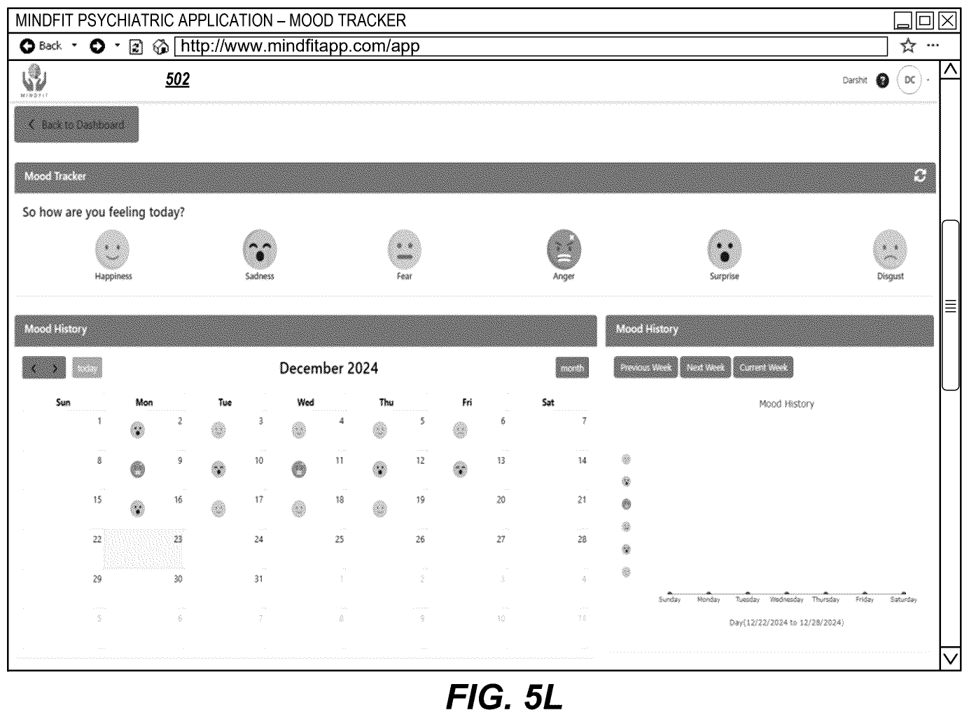

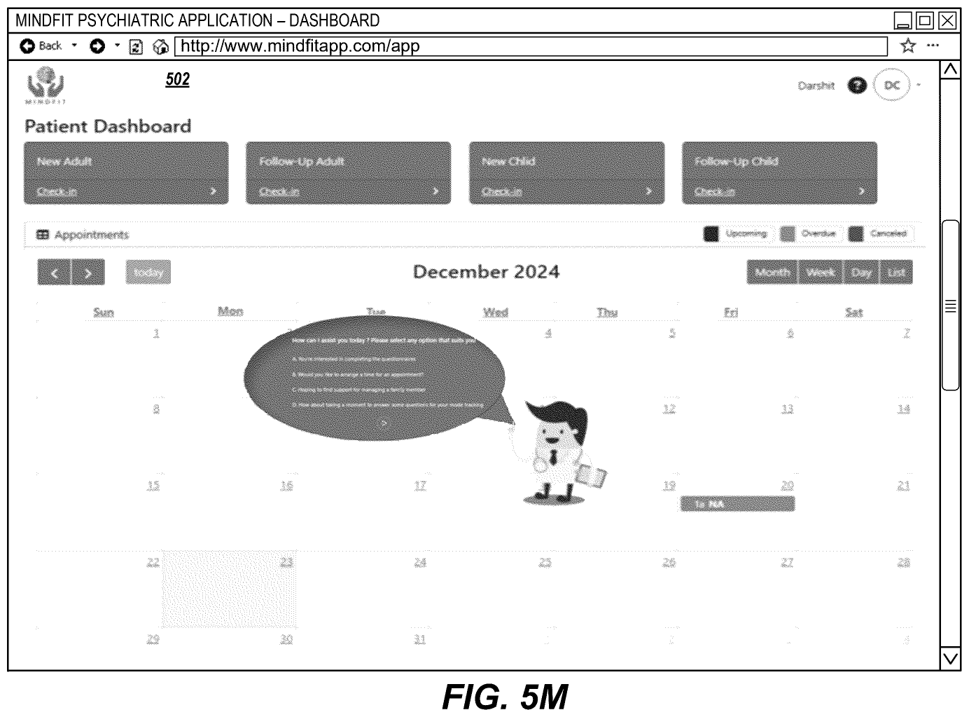

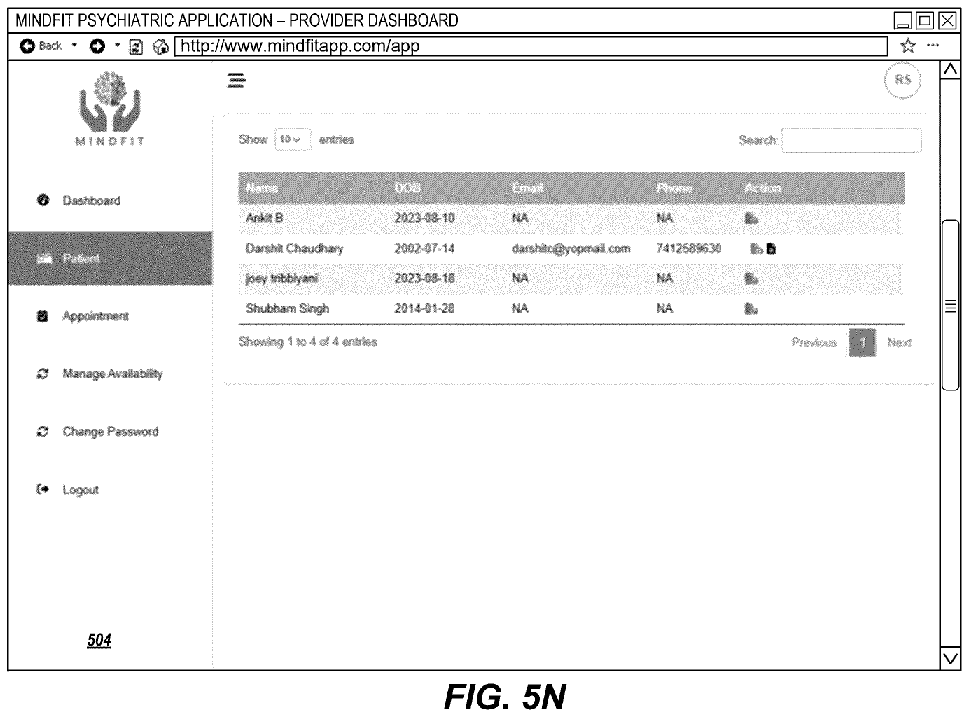

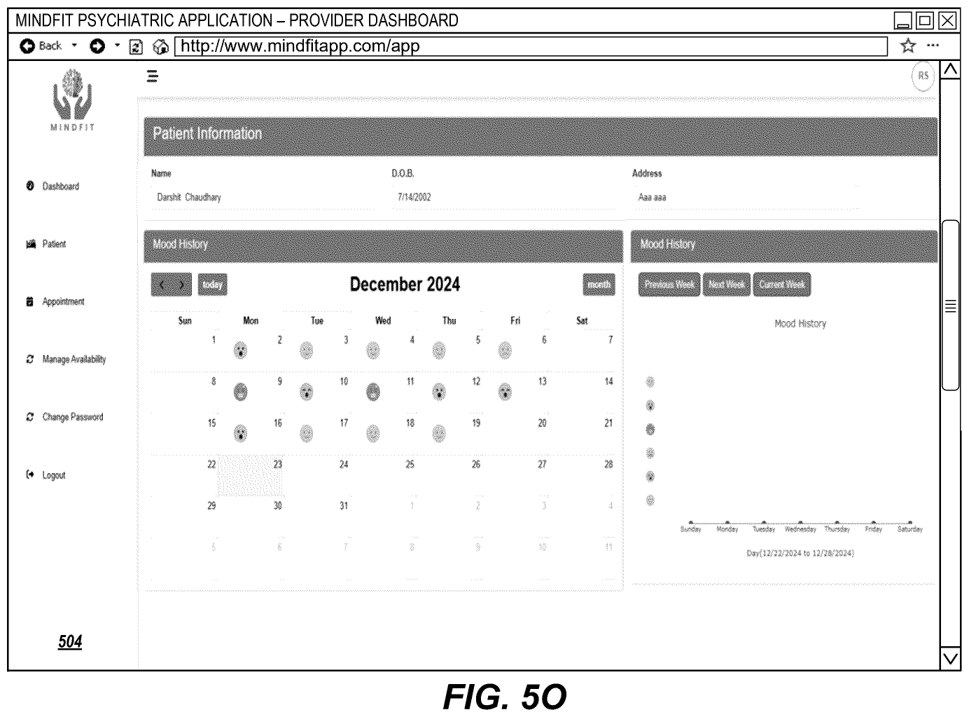

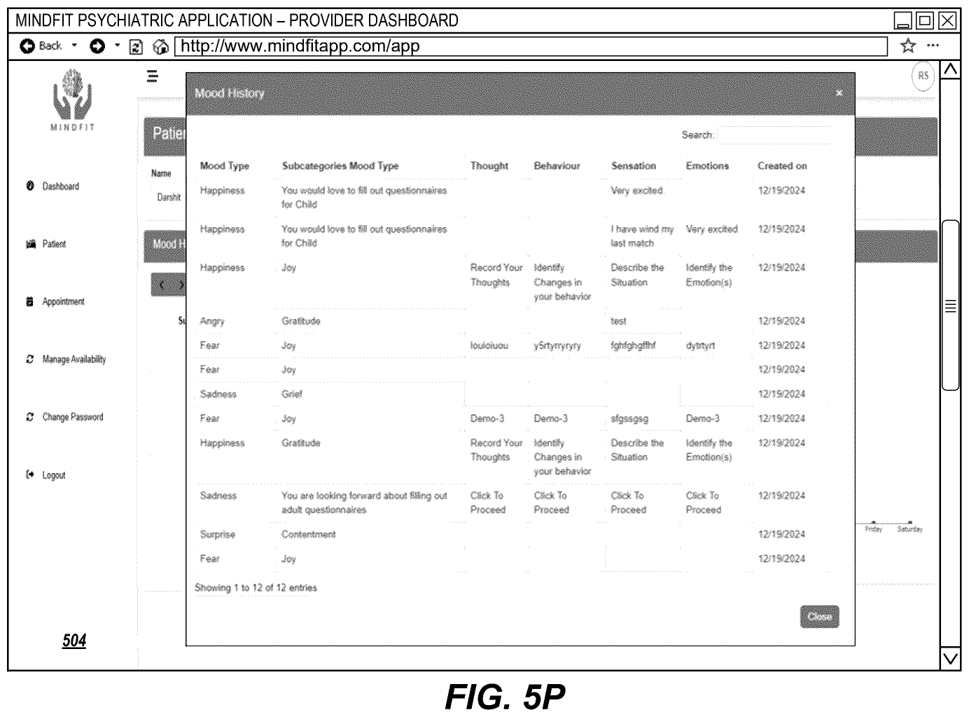

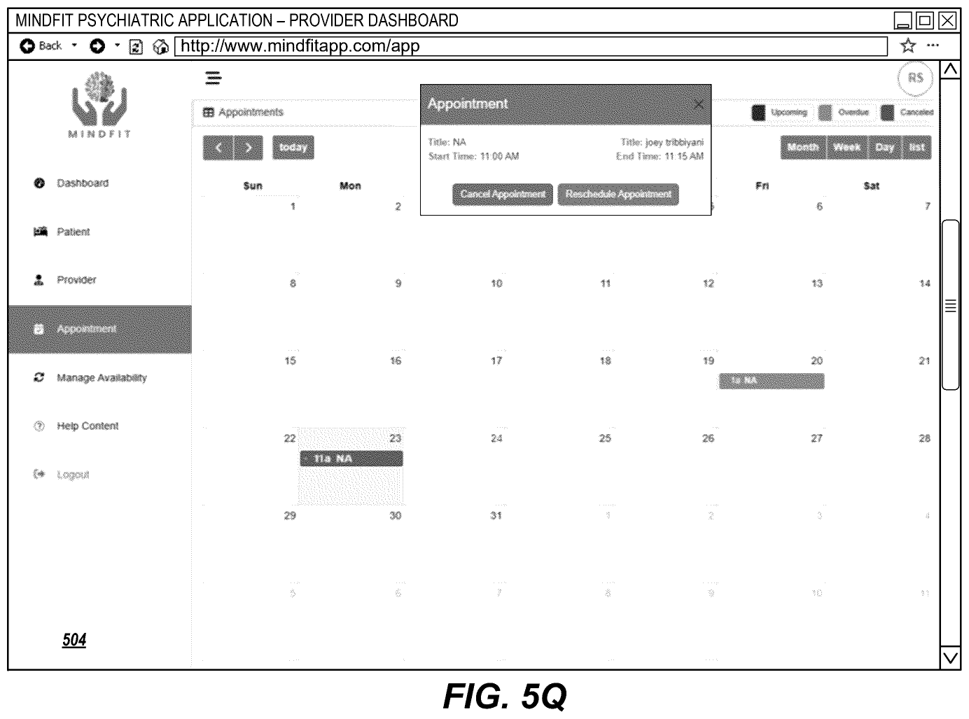

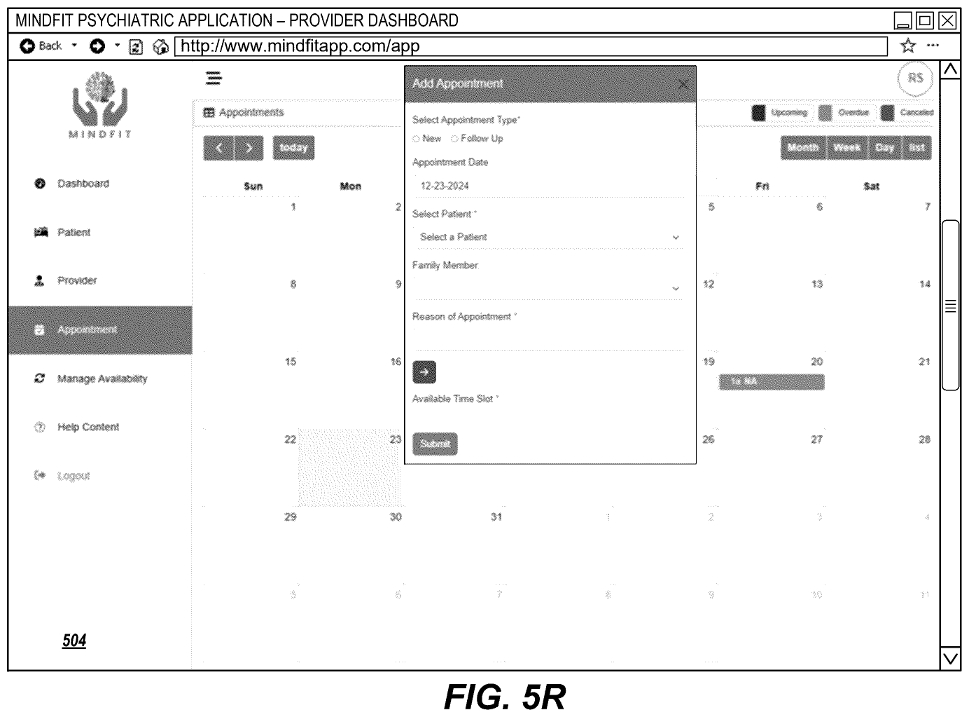

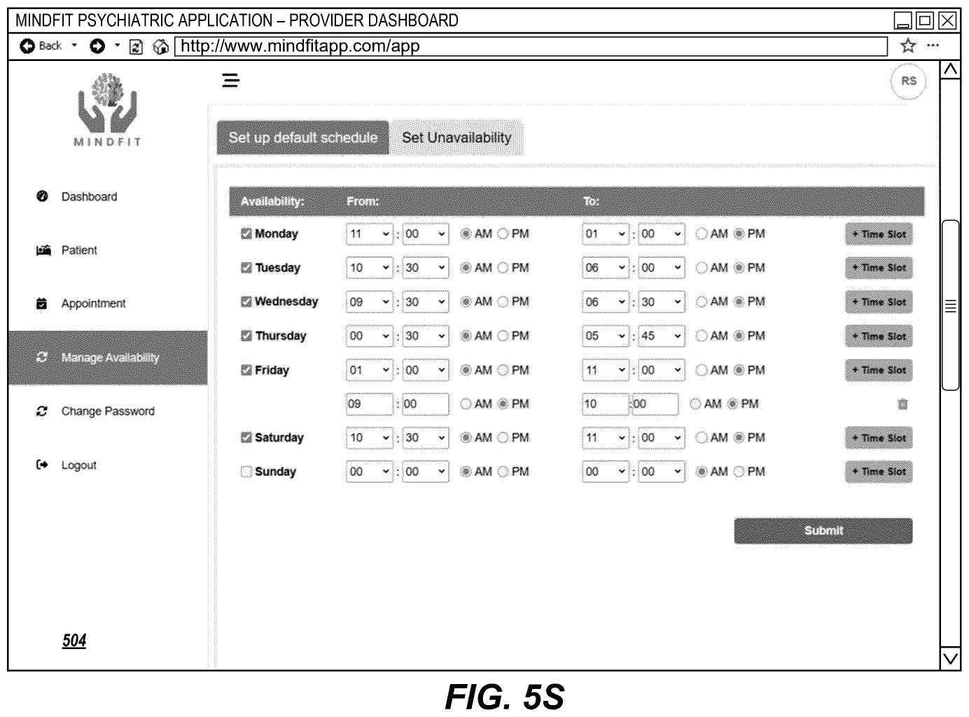

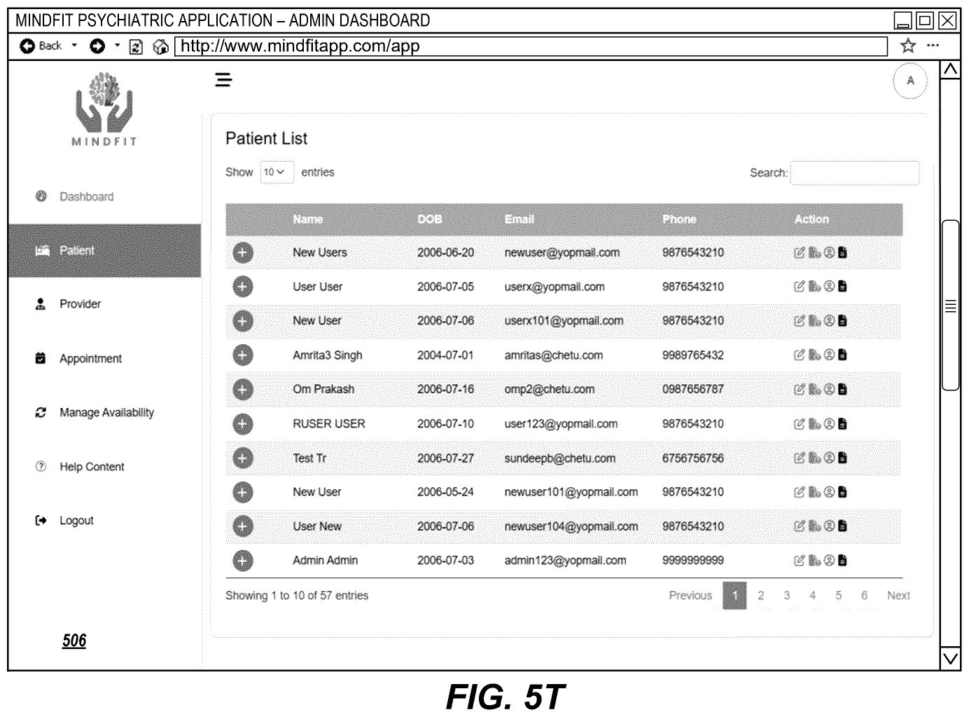

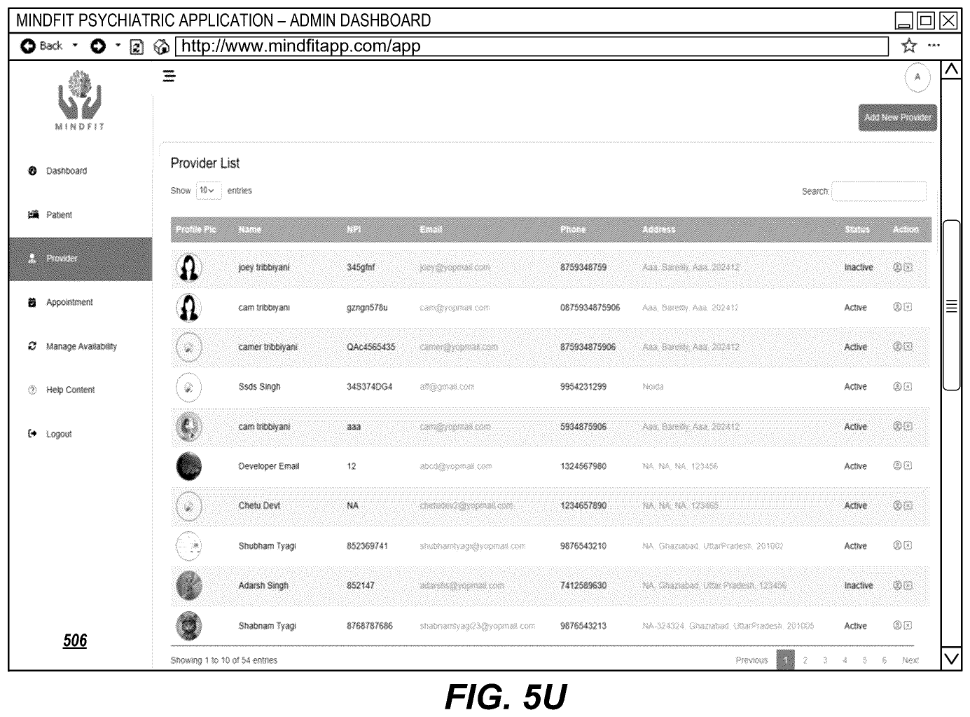

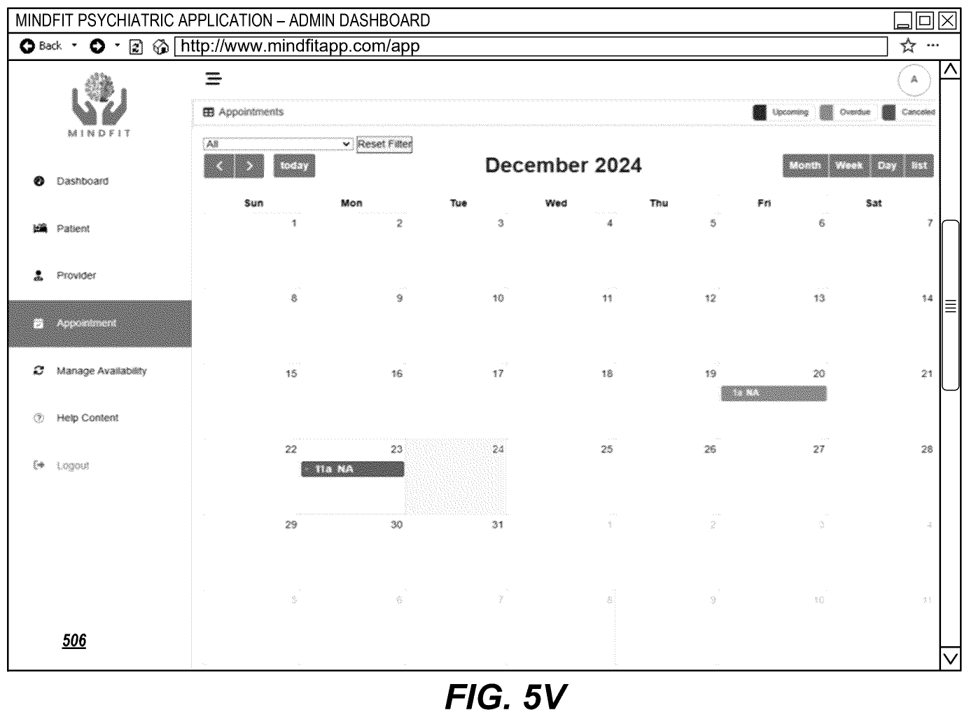

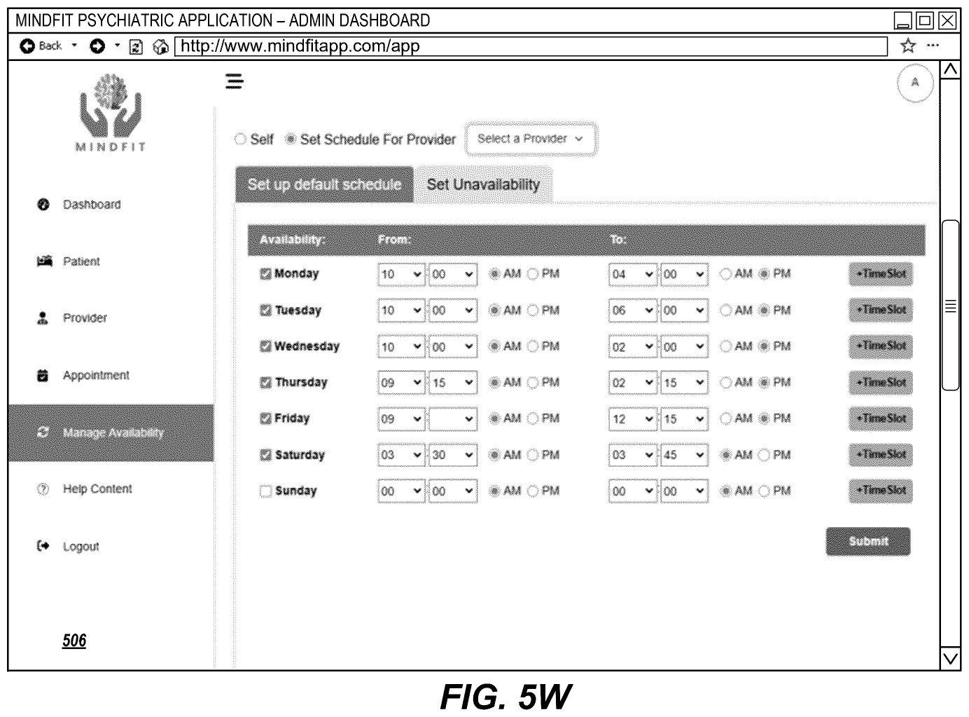

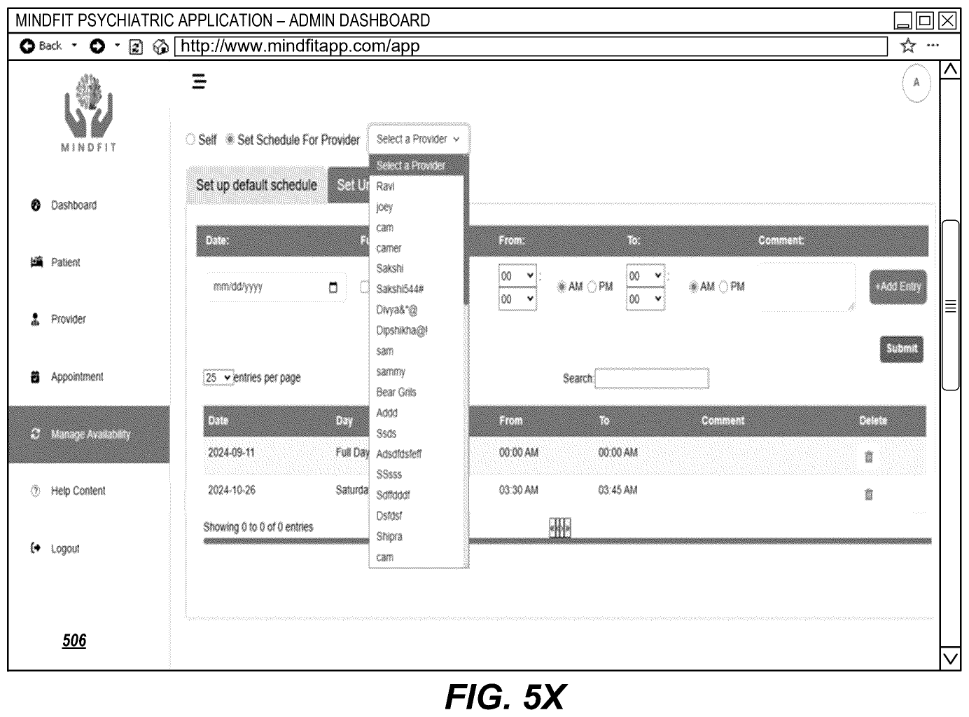

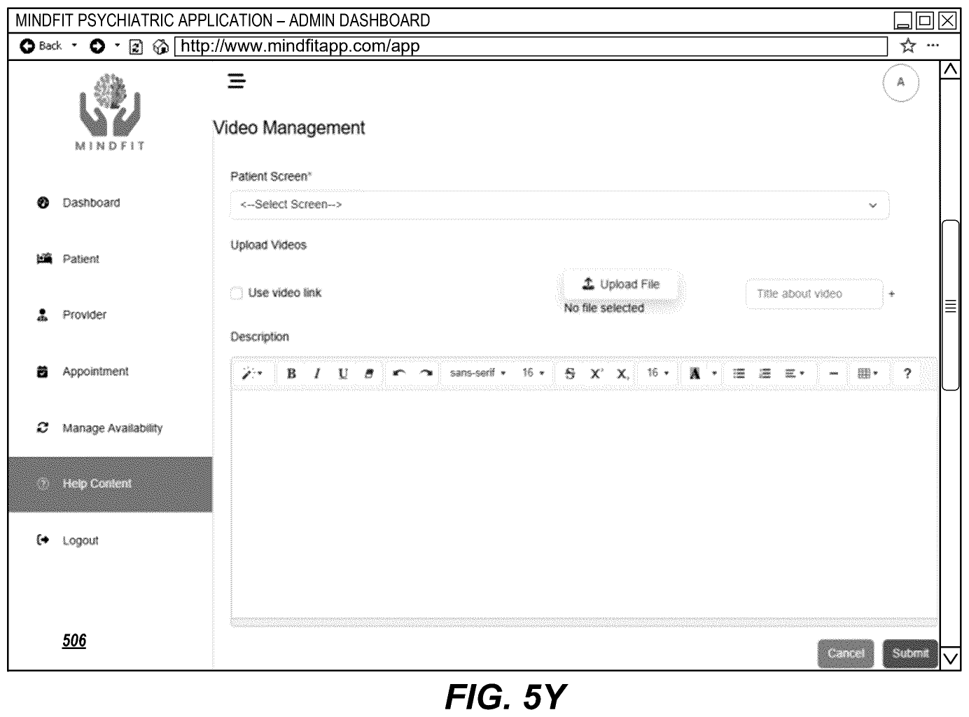

The routine 400 begins at step 402, where the standardized psychiatric interview system 100 receives psychiatric interview information 106 from a patient. The psychiatric interview information 106 may include answers to psychiatric interview questions, pathology, basic patient information, family history, medical history, and the like. In some embodiments, the psychiatric interview information 106 may be provided by a client 102 through a user interface provided by the application 108, such as portions of the patient user interface 502 shown in FIGS. 5A-5M. As may be seen in the figures, the client 102 may provide psychiatric interview information 106 at least in part in response to a number of questions/interrogatories. An exemplary set of questions/interrogatories for psychiatric interview information 106 according to various embodiments may be seen in APPENDIX I attached hereto.

The standardized psychiatric interview system 100 may then store the provided psychiatric interview information 106 in the database 112 associated with the client 102, as shown at step 404. From step 404, the routine 400 proceeds to step 406, where the standardized psychiatric interview system 100 checks if the psychiatric interview information 106 received from the patient is complete. As described herein, the client 102 may provide their psychiatric interview information 106 over multiple “sessions” of usage of the application 108. If the psychiatric interview information 106 received from the client 102 is not complete, the routine 400 returns to step 402 to await additional psychiatric interview information 106 from the client.

If, at step 406, all the necessary psychiatric interview information 106 has been received from the client 102 (patient), then the routine 400 proceeds to step 408, where the standardized psychiatric interview system 100 facilitates access to the received psychiatric interview information to the associated provider and or other authorized medical personnel. For example, as shown at step 408, the standardized psychiatric interview system 100 may format the received psychiatric interview information 106 into a “patient notes” format utilized by providers/doctors as part of a conventional EMR system. An exemplary patient notes document 114 generated from psychiatric interview information 106 may be seen in APPENDIX II attached hereto.

From step 408, the routine 400 proceeds to step 410, where the standardized psychiatric interview system 100 provides the patient notes document 114 generated from the received psychiatric interview information 106 available to the associated provider. In some embodiments, the patient notes document 114 may be displayed to the provider 116 on a computing device 104 through a user interface of the MindFit Psychiatric Application 108. In further embodiments, the patient notes document 114 may be sent to third-party EMR systems 130 associated with the client 102 for access by the provider. From step 410, the routine 400 ends.

FIGS. 5A-5Y show exemplary user interfaces 502-506 used by clients, providers, and administrators to interact with the standardized psychiatric interview system 100, according to some embodiments. For example, a patient user interface 502, as shown in FIGS. 5A-5M may be utilized by clients 102 to interact with the standardized psychiatric interview system 100. The patient user interface 502 may provide one or more of the following capabilities to the client 102 (the “user”):
Login—the login screen allows the user to provide their credentials and click on the login button. After successful login, the application shows the Patient Dashboard screen. If the user doesn't have an account in the application, the application provides the user the ability to register with the application.
Acknowledge/Term and Conditions—the user may have to accept the Terms and Conditions in order to utilize the application.
Patient Dashboard—after successfully logging in, the user is presented with the Patient Dashboard. The Patient Dashboard has multiple options to perform various operations.
Patient Questionnaire—the user is presented with a number of questions to collect psychiatric interview data. The user may review/edit data for themselves or other authorized users, e.g., children or other family members.
Appointments—user “book” appointments with providers, review and modify appointments already booked, add family members to schedule appointments for or review interview data, etc.
Past Psychiatric History
Mood Tracker—user is able to track their mood on a calendar and can see the provided mood history on a graph as well as in the calendar.
Virtual Avatar Assistant—if the user clicks the virtual avatar assistant, they are prompted with questions and options allowing them to more easily access the complete functionality of the Patient Dashboard.

A provider user interface 504, as shown in FIGS. 5N-5S may be utilized by providers 116 to interact with the standardized psychiatric interview system 100. The provider user interface 504 may provide one or more of the following capabilities to the provider 116:
Patient—provider may be shown a Patient List with certain Action buttons to perform actions on a certain patient, such as see details, review mood history, see mood history details for a particular day, etc.
Appointment—provider can see appointments with patients on a calendar, add appointments, reschedule/cancel appointments, etc.
Manage Availability—provider can set or change their availability, set their unavailability.

An administrative user interface 506, as shown in FIGS. 5T-5Y may be utilized by an administrative user of the standardized psychiatric interview system 100 to interact with the system. The administrative user interface 506 may provide one or more of the following capabilities to the administrative user:
Patient—the administrative user can see the Patient List and perform actions such as Edit, Questionnaire Summary, User Information, Mood Report, etc.
Provider—the administrative user can see the Provider List and perform actions such as Add New Provider, User Information, Inactive Provider, etc.
Appointment—the administrative user can see appointments with patients and providers on a calendar, and perform actions such as Add New Appointment, View Appointment, Delete Appointment, Reschedule Appointment, etc.
Manage Availability—the administrative user can manage Availability and Unavailability for providers.
Help Content—the administrative user can add or edit help content for the patient user interface 502.

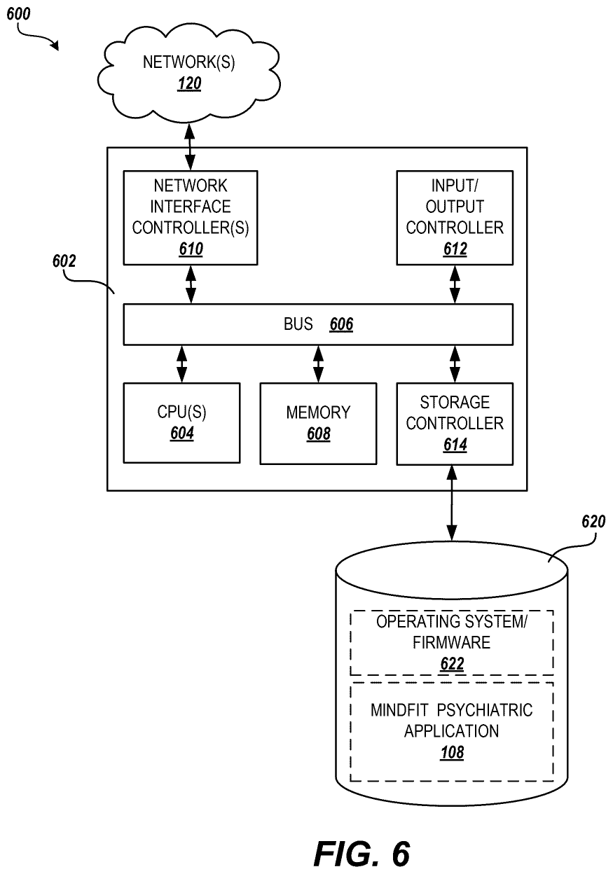

FIG. 6 shows an example computer architecture 600 for a computer 602 capable of executing software components described herein in the standardized psychiatric interview system 100. The computer architecture 600 shown in FIG. 4 illustrates a conventional server computer, workstation, desktop computer, laptop, or other computing device, and may be utilized to execute any aspects of the software components presented herein described as executing on the computing device 104, the cloud-based server(s) 110, or other computing platform. The computer 602 may include a baseboard, or “motherboard,” which is a printed circuit board to which a multitude of components or devices may be connected by way of a system bus or other electrical communication paths. In one illustrative embodiment, one or more central processing units (“CPUs”) 604 operate in conjunction with a bus 606. The CPUs 604 are standard programmable processors that perform arithmetic and logical operations necessary for the operation of the computer 602.

The CPUs 604 perform the necessary operations by transitioning from one discrete, physical state to the next through the manipulation of switching elements that differentiate between and change these states. Switching elements may generally include electronic circuits that maintain one of two binary states, such as flip-flops, and electronic circuits that provide an output state based on the logical combination of the states of one or more other switching elements, such as logic gates. These basic switching elements may be combined to create more complex logic circuits, including registers, adders-subtractors, arithmetic logic units, floating-point units, or the like.

The bus 606 provides an interface between the CPUs 604 and the remainder of the components and devices on the baseboard. The bus 606 may provide an interface to a memory 608. The memory 608 may include a random-access memory (“RAM”) used as the main memory in the computer 602. The memory 608 may further include a computer-readable storage medium such as a read-only memory (“ROM”) or non-volatile RAM (“NVRAM”) for storing basic routines that that help to startup the computer 602 and to transfer information between the various components and devices. The ROM or NVRAM may also store other software components necessary for the operation of the computer 602 in accordance with the embodiments described herein.

According to various embodiments, the computer 602 may operate in a networked environment using logical connections to remote computing devices through one or more networks, such as the network(s) 120 described above or any other networking topology known in the art that connects the computer 602 to other, remote computers. The bus 606 may include functionality for providing network connectivity through one or more network interface controllers (“NICs”) 610, such as a gigabit Ethernet adapter. It should be appreciated that any number of NICs 610 may be present in the computer 602, connecting the computer to other types of networks and remote computer systems beyond those described herein.

The computer 602 may be connected to a mass storage device 620 that provides non-volatile storage for the computer. The mass storage device 620 may store system programs, application programs, other program modules, and data, which are described in greater detail herein. The mass storage device 620 may be connected to the computer 602 through a storage controller 614 connected to the bus 606. The mass storage device 620 may consist of one or more physical storage units. The storage controller 614 may interface with the physical storage units through a serial attached SCSI (“SAS”) interface, a serial advanced technology attachment (“SATA”) interface, a fiber channel (“FC”) interface, or other standard interface for physically connecting and transferring data between computers and physical storage devices.

The computer 602 may store data on the mass storage device 620 by transforming the physical state of the physical storage units to reflect the information being stored. The specific transformation of physical state may depend on various factors, in different implementations of this description. Examples of such factors may include, but are not limited to, the technology used to implement the physical storage units, whether the mass storage device 620 is characterized as primary or secondary storage, or the like. For example, the computer 602 may store information to the mass storage device 620 by issuing instructions through the storage controller 614 to alter the magnetic characteristics of a particular location within a magnetic disk drive unit, the reflective or refractive characteristics of a particular location in an optical storage unit, or the electrical characteristics of a particular capacitor, transistor, or other discrete component in a solid-state storage unit. Other transformations of physical media are possible without departing from the scope and spirit of the present description, with the foregoing examples provided only to facilitate this description. The computer 602 may further read information from the mass storage device 620 by detecting the physical states or characteristics of one or more particular locations within the physical storage units.

The mass storage device 620 may store an operating system 622 utilized to control the operation of the computer 602. According to some embodiments, the operating system comprises the LINUX operating system. According to another embodiment, the operating system comprises the WINDOWS® SERVER operating system from MICROSOFT Corporation of Redmond, Washington. According to further embodiments, the operating system may comprise the UNIX or SOLARIS operating systems. It should be appreciated that other operating systems may also be utilized. The mass storage device 620 may store other system or application program modules and data utilized by the computer 602, such as the MindFit Psychiatric Application 108 implementing functionality and user interfaces described herein for or providing a streamlined and standardized psychiatric interview process.

In some embodiments, the mass storage device 620 may be encoded with computer-executable instructions that, when loaded into the computer 602, may transform the computer from a general-purpose computing system into a special-purpose computer capable of implementing the embodiments described herein. These computer-executable instructions transform the computer 602 by specifying how the CPUs 604 transition between states, as described above. According to some embodiments, the mass storage device 620 may store computer-executable instructions that, when executed by the computer 602, perform the routine 400 described herein for providing a streamlined and standardized psychiatric interview process. In further embodiments, the computer 602 may have access to other computer-readable storage medium in addition to or as an alternative to the mass storage device 620.

The computer 602 may also include an input/output controller 612 for receiving and processing input from a number of input devices, such as a keyboard, a mouse, a touchpad, a touch screen, an electronic stylus, or other type of input device. Similarly, the input/output controller 612 may provide output to a display device, such as a computer monitor, a flat-panel display, a digital projector, a printer, a plotter, or other type of output device. It will be appreciated that the computer 602 may not include all of the components shown in FIG. 6, may include other components that are not explicitly shown in FIG. 6, or may utilize an architecture completely different than that shown in FIG. 6. For example, the processor(s) 604, memory 608, mass storage devices 620, and NIC(s) 610 of the computer architecture 600 may represent components of a System-on-a-Chip (“SoC”) integrated circuit utilized in a mobile device or smartphone, virtualized resources from any number of server computers or computing devices, or generic processing resources, storage resources, and communication resources of a cloud-based computing system, with the bus 606 representing communication interlinks between the processing, storage, communication, and other computing resources in the cloud-based computing system. It is intended that all such computing architectures be included within the scope of this application.

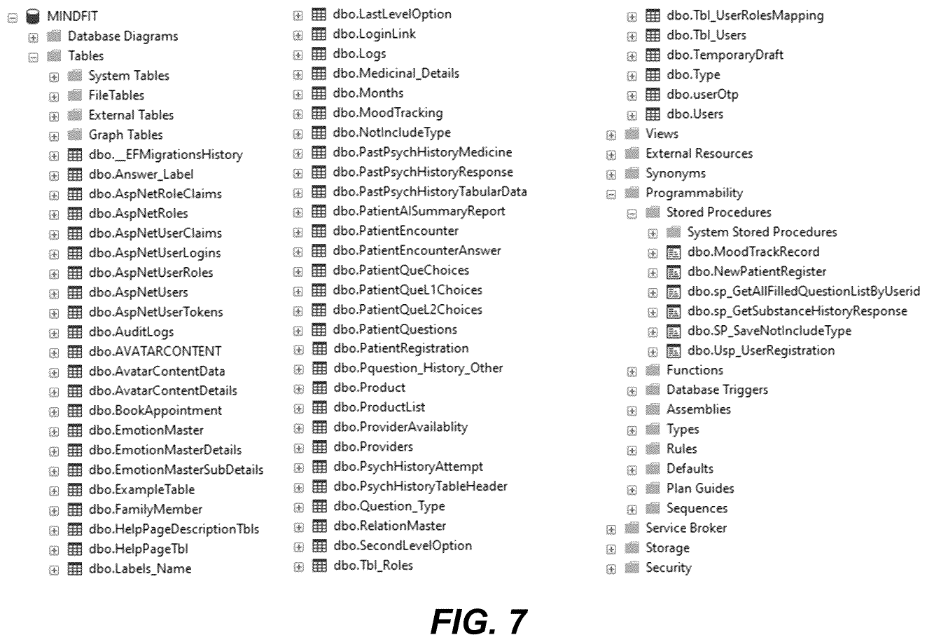

FIG. 7 is an inventory list of exemplary database tables and stored procedures for storing data supporting the standardized psychiatric interview system 100, according to embodiments presented herein. The database tables and/or stored procedures may be part of the definition of a RDS stored in the database 112 of the standardized psychiatric interview system 100, for example.

Based on the foregoing, it will be appreciated that technologies for or providing a streamlined and standardized psychiatric interview process are described herein. The above-described embodiments are merely possible examples of implementations set forth for a clear understanding of the principles of the present disclosure. Many variations and modifications may be made to the above-described embodiments without departing substantially from the spirit and principles of the present disclosure. All such modifications and variations are intended to be included within the scope of the present disclosure, and all possible claims to individual aspects or combinations and sub-combinations of elements or steps are intended to be supported by the present disclosure.

The logical steps, functions or operations described herein as part of a routine, method or process may be implemented (1) as a sequence of processor-implemented acts, software modules or portions of code running on a microcontroller, computing device, or other computer system and/or (2) as interconnected machine logic circuits or circuit modules within the microcontroller, computing device, or other computer system. The implementation is a matter of choice dependent on the performance and other requirements of the system. Alternate implementations are included in which steps, operations or functions may not be included or executed at all, may be executed out of order from that shown or discussed, including substantially concurrently or in reverse order, depending on the functionality involved, as would be understood by those reasonably skilled in the art of the present disclosure.

It will be further appreciated that conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more particular embodiments or that one or more particular embodiments necessarily include logic for deciding, with or without user input or prompting, whether these features, elements and/or steps are included or are to be performed in any particular embodiment.

What is claimed is:

1. A method of conducting a standardized psychiatric interview with a client online, the method comprising steps of:
receiving, by a standardized psychiatric interview system, psychiatric interview information from a client over a network;
storing, by the standardized psychiatric interview system, the received psychiatric interview information in a database;
determining, by the standardized psychiatric interview system, whether the received psychiatric interview information for the client is complete;
upon determining that the received psychiatric interview information for the client is not complete, repeating, by the standardized psychiatric interview system, the receiving, storing, and determining steps;
upon determining that the psychiatric interview information for the client is complete, formatting, by the standardized psychiatric interview system, the received psychiatric interview information for the client into a standardized patient notes document; and
providing, by the standardized psychiatric interview system, the standardized patient notes document for the client to a provider associated with the client over the network.

2. The method of claim 1, wherein the psychiatric interview information comprises responses to a plurality of interrogatories provided to the client on a computing device connected to the standardized psychiatric interview system over the network.

3. The method of claim 2, wherein the psychiatric interview information comprises one or more answers to psychiatric interview questions, pathology, basic patient information, family history, and medical history.

4. The method of claim 2, wherein the standardized psychiatric interview system is implemented on cloud-based computing resources connected to the computing device of the client over the Internet.

5. The method of claim 1, wherein the standardized patient notes document is formatted into a standard patient notes format utilized as part of a conventional electronic medical records (EMR) system.

6. The method of claim 5, further comprising the steps of:
providing, by the standardized psychiatric interview system, the standardized patient notes document to the EMR system connected to the standardized psychiatric interview system over the network.

7. The method of claim 1, wherein the client provides complete psychiatric interview information to the standardized psychiatric interview system and the formatted standardized patient notes document is provided to the provider before a scheduled appointment of the client with the provider.

8. A non-transitory computer-readable medium containing processor-executable instructions that, when executed by a processor of a standardized psychiatric interview system, cause the standardized psychiatric interview system to:
receive psychiatric interview information from a client over a network;
store the received psychiatric interview information in a database;
determine whether the received psychiatric interview information for the client is complete;
upon determining that the received psychiatric interview information for the client is not complete, repeat the receiving, storing, and determining;
upon determining that the psychiatric interview information for the client is complete, format the received psychiatric interview information for the client into a standardized patient notes document; and
provide the standardized patient notes document for the client to a provider associated with the client over the network.

9. The non-transitory computer-readable medium of claim 8, wherein the psychiatric interview information comprises responses to a plurality of interrogatories provided to the client on a computing device connected to the standardized psychiatric interview system over the network.

10. The non-transitory computer-readable medium of claim 9, wherein the psychiatric interview information comprises one or more answers to psychiatric interview questions, pathology, basic patient information, family history, and medical history.

11. The non-transitory computer-readable medium of claim 8, wherein the standardized patient notes document is formatted into a standard patient notes format utilized as part of a conventional electronic medical records (EMR) system.

12. The non-transitory computer-readable medium of claim 11, containing further processor-executable instructions that cause the standardized psychiatric interview system to:
provide the standardized patient notes document to the EMR system connected to the standardized psychiatric interview system over the network.

13. The non-transitory computer-readable medium of claim 8, wherein the client provides complete psychiatric interview information to the standardized psychiatric interview system and the formatted standardized patient notes document is provided to the provider before a scheduled appointment of the client with the provider.

14. A standardized psychiatric interview system comprising:
a database; and
a processor operably connected to the database and configured to, upon receiving psychiatric interview information from a client over a network:
store the received psychiatric interview information in the database,
determine whether the received psychiatric interview information for the client is complete,
upon determining that the psychiatric interview information for the client is complete, format the received psychiatric interview information for the client into a standardized patient notes document, and
provide the standardized patient notes document for the client to a provider associated with the client over the network.

15. The standardized psychiatric interview system of claim 14, wherein the psychiatric interview information comprises responses to a plurality of interrogatories provided to the client on a computing device associated with the client connected to the standardized psychiatric interview system over the network.

16. The standardized psychiatric interview system of claim 15, wherein the psychiatric interview information comprises one or more answers to psychiatric interview questions, pathology, basic patient information, family history, and medical history.

17. The standardized psychiatric interview system of claim 15, wherein the database and processor comprise cloud-based computing resources connected to the computing device associated with the client over the Internet.

18. The standardized psychiatric interview system of claim 14, wherein the standardized patient notes document is formatted into a standard patient notes format utilized as part of a conventional electronic medical records (EMR) system.

19. The standardized psychiatric interview system of claim 18, wherein the processor is further configured to provide the standardized patient notes document to the EMR system connected to the standardized psychiatric interview system over the network.

20. The standardized psychiatric interview system of claim 14, wherein the client provides complete psychiatric interview information to the standardized psychiatric interview system and the formatted standardized patient notes document is provided to the provider before a scheduled appointment of the client with the provider.
